(19) United States
(12) Patent Application Publication
(10) Pub. No.: US 2002/0168287 A1
(43) Pub. Date: Nov. 14, 2002

(54) Method and apparatus for rapidly sterilizing irregularly-shaped objects

(76) Inventors: Richard Eckhardt, Arlington, MA (US); Geoffrey H. Jenkins, Wellesley Hills, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/107,692

(22) Filed: Mar. 27, 2002

Related U.S. Application Data
(60) Provisional application No. 60/279,053, filed on Mar. 27, 2001.

(51) Int. Cl. A61L 2/10; A61L 2/00

(52) U.S. Cl. 422/24; 422/22; 422/28; 250/455.11

(57) ABSTRACT

Methods and apparatus for sterilizing or disinfecting irregularly-shaped objects. One embodiment of the invention is directed to an apparatus including a substantially light-tight housing, at least one ultraviolet light-emitting lamp disposed within the housing, and a drum to change the orientation of the object in the housing so that light emitted by the at least one lamp contacts substantially all of the exterior surfaces of the object. Another embodiment of the invention is directed to a batch process that includes enclosing an object within a substantially light-tight housing, and changing the orientation of the object in the housing while irradiating the object with ultraviolet light so that the ultraviolet light contacts substantially all exterior surfaces of the object. A further embodiment of the invention is directed to an apparatus including a housing having a light seal to substantially prevent light from penetrating the housing, at least one ultraviolet light-emitting lamp disposed within the housing, and a mechanism for moving the object into the housing, wherein the mechanism forms at least part of the light seal. Another embodiment of the invention is directed to a method including acts of introducing an object into a housing, causing the object to move through the housing, detecting the object with a sensor, and activating a lamp based on a detection of the object with the sensor, the lamp illuminating the object as it moves through the housing.

### PRIORITY CLAIM

This application claims the benefit, under 35 U.S.C. §119(e), of the filing date of U.S. provisional application serial No. 60/279,053 entitled “Method and Apparatus for Rapidly Sterilizing Irregular-Shaped Objects,” filed Mar. 27, 2001, which is incorporated herein by reference.

### FIELD OF THE INVENTION

The present invention relates generally to the field of sterilization or disinfection systems and methods.

### BACKGROUND OF THE INVENTION

A number of objects used in everyday life, particularly those used in medical and hygienic applications, can serve as a transport mechanism for disease causing microorganisms. Objects that are handled or breathed-on by different people, or that come in contact with surfaces contaminated by other people or animals, can themselves become contaminated. If these objects then contact another person, they can transmit diseases. Even the hands and clothing of medical or healthcare personnel can transmit diseases.

This contamination problem is particularly acute with objects used in medical facilities or for hygienic applications at home or in public facilities, as objects in these settings have a much higher probability of contacting infected people or surfaces. Some medical devices are designed to be placed in contact with diseased patients. If they are not sterilized between use on different patients, they can serve as the vector to transmit the disease from one person to the next. Some medical devices, such as thermometers and otoscopes, are well recognized as disease vectors and are commonly used with disposable elements or covers to prevent the transmission of microorganisms. Other medical devices, such as stethoscopes, blood pressure measuring devices, blood oxygen level probes, and EKG equipment, are typically not used with disposable covers because the covers are more difficult to implement. Some of these devices can be manufactured in disposable forms, but this generally results in a compromise in quality and/or cost. Manual sterilization with disinfectant chemicals is sometimes done, but this is time consuming and not performed as often as is desirable. The hands and clothing of healthcare workers are often sterilized by washing, but this can be inconvenient, time consuming, and ineffective. Equipment in the non-medical applications listed above are typically only occasionally and haphazardly sterilized as part of the routine cleaning.

Thus, there is a need for an improved technique for sterilizing medical, hygienic, and other devices. The use of ultraviolet (UV) light for sterilization is known in the art, and has a number of potential advantages. Chemicals and heat, which can be damaging and/or time consuming, do not need to be applied for successful sterilization or disinfection when UV light is used. However, sterilization or disinfection using UV light can be difficult when the object to be sterilized or disinfected is irregularly-shaped or flexible, since it can be difficult to assure sufficient exposure at all points on the surface for these objects.

U.S. Pat. No. 5,597,597, which issued to Paul B. D. Newman on Jan. 28, 1997, describes a sterilizing unit that is exemplary of prior art approaches for sterilization using UV light. According to one disclosed embodiment, food to be irradiated within a chamber is introduced into a rotatable mesh drum by means of a conveyor. Spirally arranged baffles guide the food in a spiral fashion from one open end of the drum to the other, while the food is irradiated by light sources disposed outside of the drum. After leaving the irradiating chamber, the food is transferred from the drum onto a second conveyor. Applicants herein have discovered that this approach presents drawbacks when applied in a clinical or commercial setting. For example, conveyor systems can be large and are generally not conducive to being sealed against light exposure. The system described above, used in a factory setting, has a drum within a much larger chamber, in which UV lights are mounted. The chamber and conveyor contribute to the large size of the unit. Further, the unit does not employ light protection measures.

As should be appreciated from the foregoing, there exists a need for improved systems and methods of sterilization or disinfection.

### SUMMARY OF THE INVENTION

One embodiment of the invention is directed to an apparatus for sterilizing or disinfecting an object having exterior surfaces. The apparatus comprises a substantially light-tight housing, at least one ultraviolet light-emitting lamp disposed within the housing, and a drum to change the orientation of the object in the housing so that light emitted by the at least one lamp contacts substantially all of the exterior surfaces of the object.

Another embodiment of the invention is directed to an apparatus for sterilizing or disinfecting an object having exterior surfaces. The apparatus comprises a substantially light-tight housing, at least one ultraviolet light-emitting lamp disposed within the housing, and means for changing the orientation of the object in the housing so that light emitted by the at least one lamp contacts substantially all of the exterior surfaces of the object.

A further embodiment of the invention is directed to a batch process for sterilizing or disinfecting an object having exterior surfaces, comprising acts of enclosing the object within a substantially light-tight housing, and changing the orientation of the object in the housing while irradiating the object with ultraviolet light so that the ultraviolet light contacts substantially all of the exterior surfaces of the object.

Another embodiment of the invention is directed to a method for sterilizing or disinfecting an object. The method comprises acts of introducing the object into a housing, causing the object to move through the housing, detecting the object with a sensor, and activating a lamp based on a detection of the object with the sensor, the lamp illuminating the object as it moves through the housing.

A further embodiment of the invention is directed to an apparatus for sterilizing or disinfecting an object. The apparatus comprises a housing having a light seal to substantially prevent light from penetrating the housing, at least one ultraviolet light-emitting lamp disposed within the housing, and a mechanism for moving the object into the housing, wherein the mechanism forms at least part of the light seal.

### BRIEF DESCRIPTION OF THE DRAWINGS

FIGS. 1A and 1B are diagrams respectively illustrating a front cross-sectional view and a side cross-sectional view of a sterilizer/disinfector according to one embodiment of the invention; and

FIGS. 2A and 2B are diagrams respectively illustrating a front cross-sectional view and a side cross-sectional view of a sterilizer/disinfector according to another embodiment of the invention.

### DETAILED DESCRIPTION

The present invention is directed to a method and apparatus for sterilizing or disinfecting objects using ultraviolet light. An object to be sterilized or disinfected may include any irregularly shaped object, such as an object that is not straight, uniform, or symmetrical, that may come into contact with a carrier of microorganisms (e.g., a person or animal). Examples of such objects are pulse oximeters, otoscopes, blood pressure cuffs, EKG connectors and wires, stethoscopes, and other devices used by used by doctors, nurses, and other medical or healthcare personnel. Other examples are the equipment used in alternative medicine clinics, chiropractic offices, dental offices, nursing homes, veterinary clinics, commercial businesses such as hair or nail salons, and commercial and public food service establishments. The apparatus may be light-tight to prevent accidental UV exposure to a user, may be sized for a clinical or commercial setting, and may be suitable for sterilizing or disinfecting irregularly-shaped objects.

Batch Process Sterilizer/Disinfector

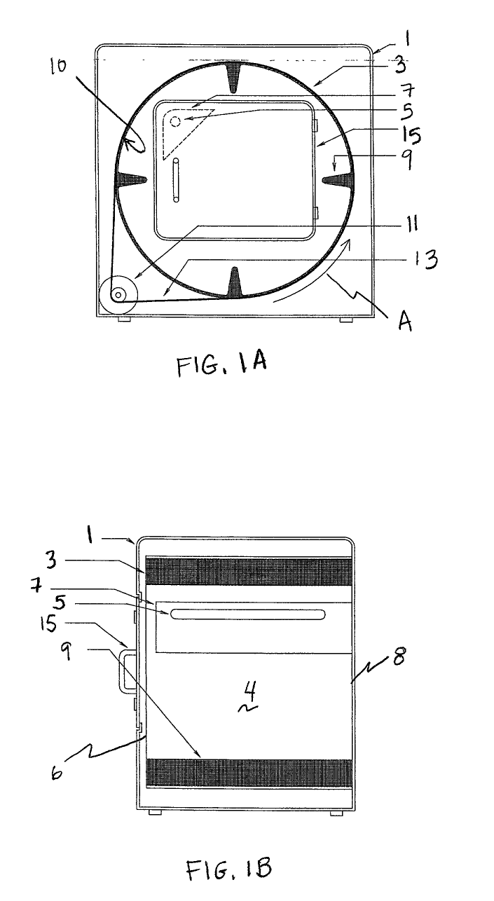

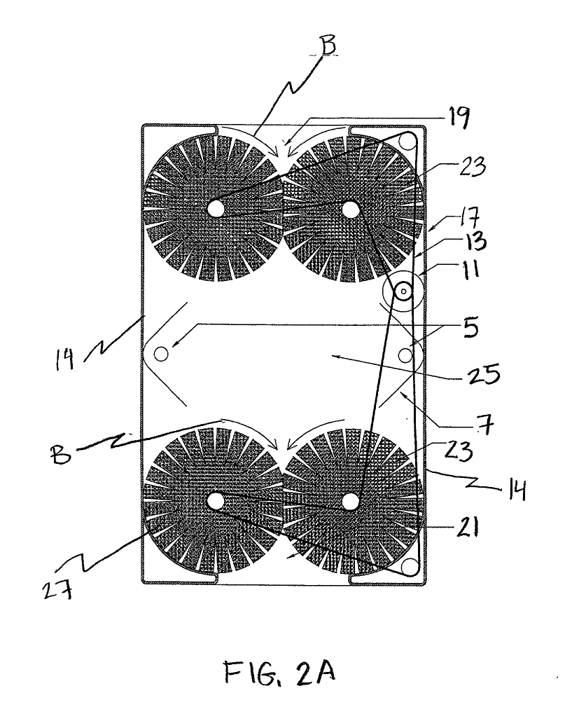

In accordance with one illustrative embodiment of the invention, an apparatus is provided that may sterilize or disinfect objects of varied shapes using a batch process. FIGS. 1A-1B illustrate one example of such an apparatus. As shown in FIG. 1A, a housing 1 contains a drum 3 that defines a sterilization chamber 4. Drum 3 may be any structure that moves or rotates to tumble, agitate, or otherwise move an object, and may have a shape that is, e.g., cylindrical, spherical, square, or polygonal. A door 15, which may alternatively be implemented as any port allowing an object to be loaded and unloaded, provides access to sterilization chamber 4 from outside of housing 1. A lamp 5, which emits light for sterilization or disinfection, is provided within sterilization chamber 4. Although lamp 5 is shown mounted on a back wall 8 of housing 1, lamp 5 may be mounted on a front wall 6 of housing 1, on drum 3, or in any other location that allows for illumination of the object and/or sterilization chamber 4. Reflector 7 may be disposed about lamp 5 to direct the light emitted from lamp 5 towards the object.

Lamp 5 may be any light source that emits light capable of sterilization or disinfection. For example, lamp 5 may be a UV light source such as a mercury vapor lamp, a xenon flash lamp, a continuous arc lamp, UV light emitting diodes (LEDs), a UV laser, or any other solid state or non-solid state UV light-emitting device. The lamp may emit narrow spectrum light (e.g., a line spectrum) or broad spectrum light. Broad spectrum light may include, e.g., UVA, UVB, and UVC light, or UV light accompanied by light from another portion of the electromagnetic spectrum. For example, the emission of both UV and visible light from lamp 5 may enhance the effectiveness of the light source, as the sensitivity of different microorganisms to light varies with the wavelength of the light. Light can be generated by lamp 5 in flashes or as continuous radiation. If a flash lamp or other non-continuous lamp is used, it may be flashed repeatedly during the sterilization process to assure complete exposure of the object(s) to be sterilized. It should be appreciated that though a single lamp 5 is described and illustrated, one or more light sources may be used.

Drum 3 is coupled to a motor 11 via a drive belt 13. Motor 11 may be controlled to cause drive belt 13 to move drum 3 in the direction of arrow A. According to the illustrative embodiment of FIGS. 1A-1B, drum 3 rotates counterclockwise, however drum 3 may also or alternatively rotate clockwise. Further, complete rotation is not necessary, as drum 3 may rotate partially or move in a back-and-forth motion (i.e., a reciprocating motion). Drum 3 includes paddles 9 disposed on an interior wall 10 thereof. The paddles may be used to agitate or tumble an object or objects placed within sterilization chamber 4. For example, if drum 3 is rotated, an object placed in drum 3 will be carried upwards (in the direction of arrow A) in sterilization chamber 4 by one of paddles 9 until the object falls, by force of gravity, from the paddle. This lifting and dropping motion will be repeated until the rotation of the drum is stopped by the turning off of motor 11. The motion of drum 3, and the continuous repositioning of the object(s) in drum 3 that results from the motion, allows all surfaces of an object, even if irregularly-shaped, to be exposed to lamp 5.

Although motor 11 and drive belt 13 are described as providing movement to drum 3, it should be appreciated that the invention is not limited to the use of a motor and drive belt. For example, other sources of rotational energy, e.g., a crank, may be used in place of motor 11, and other mechanisms for coupling the energy to drum 3, e.g., chains, gears, or rollers, may be used in place of drive belt 13. Further, although paddles 9 are described as providing agitation to the object being sterilized/disinfected, the invention is not limited to use of a paddles for movement of the object, as other structures, e.g., clips or pegs, may be provided for moving, stirring, or tumbling the objects.

Housing 1 may form a light-tight enclosure to prevent UV light exposure to an operator. If the sterilizer/disinfector is used in a clinical setting, the penetration of UV light through housing 1 could have potentially harmful effects on nearby doctors, nurses, or patients. Accordingly, a seal 12, e.g., a gasket or interleaved structures, may be provided around the periphery of the opening for door 15, to prevent light penetration in the area between housing 1 and door 15. An interlock switch may also be provided to turn off lamp 5 when door 15 is opened, thereby preventing accidental exposure of an operator to light. The interlock switch may be activated by the door 15, a latch of the door, or the detection of external light. Activation of the interlock switch may also turn off motor 11 to stop movement of the drum when the door is opened. In another example, a timer may be provided to turn on or off lamp 5 or motor 11.

Portions of the interior of housing 1, including drum 3, paddles 9, the interior of door 15, and back wall 8 of housing 1, may be formed, for example, from reflective or cushioned materials. For example, reflective materials such as metal or reflective plastic may be used on the surfaces to reduce energy loss from sterilization chamber 4 and distribute light within sterilization chamber 4. Cushioned materials such as foam or rubber may be used to prevent damage to an object being sterilized or disinfected and reduce the noise generated by sterilization chamber 4. Drum 3 may have a continuous surface and may be substantially impenetrable to light.

The length of time required for sterilization depends on the amount of light generated by lamp 5, the reflective efficiency of sterilization chamber 4, and the surface area of the object(s) to be sterilized or disinfected. Typically, for a sterilization process, an object must be exposed to approximately 10 millijoules of UV light energy per square centimeter. Greater or lesser amounts may be required depending on the spectrum of the UV light, characteristics of the object, and environmental conditions such as temperature. A sterilization process for all surfaces of an object using the sterilizer/disinfector described above may be completed in less than a minute using a high power UV source, such as a xenon flash lamp operating at a high repetition rate, and a small sterilization chamber. Larger, or lower powered units may take tens of minutes or longer.

The sterilizer/disinfector described above may be built in a range of sizes, and may be sized to fit on a tabletop. It is not necessary that the shape of sterilization chamber 4 be cylindrical or that the axis of rotation of drum 3 be horizontal. For example, the axis of rotation may be vertical. The movement of the objects to be sterilized can be provided by the movement of an actuator internal to sterilization chamber, rather than, or in addition to, the movement of the drum. As should be appreciated, the size, shape, and configuration of the sterilizer/disinfector may be chosen according to the objects to be sterilized/disinfected.

Continuous Process Sterilizer/Disinfector

In accordance with another illustrative embodiment of the invention, an apparatus is provided to sterilize or disinfect objects of varied shapes using a continuous process. A continuous process in one in which objects are sterilized in series, such that an object may be loaded or unloaded while another object is sterilized/disinfected. The apparatus may include one or more moving light seals to allow an object to enter or exit the chamber while substantially preventing light from leaving the chamber. Thus, objects may be loaded or unloaded while the sterilizer/disinfector is in operation. The apparatus according to this illustrative embodiment may be particularly useful when used with long and/or flexible objects, such as the connectors and wires used for EKG machines.

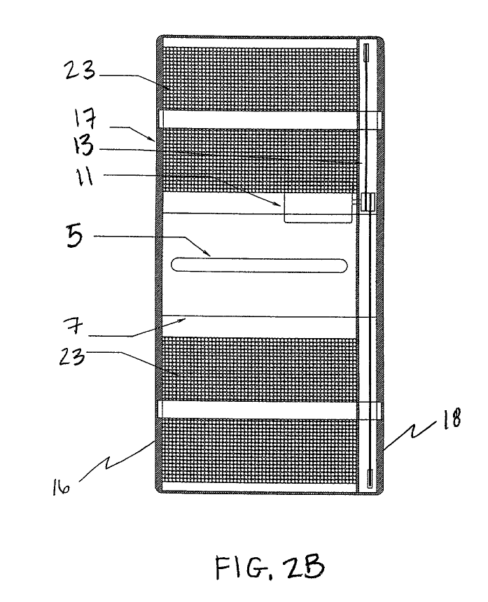

FIGS. 2A-2B illustrate one example of a sterilizing/disinfecting apparatus that uses a continuous process. As shown in FIG. 2A, a housing 17 contains rollers 23 that define a sterilization chamber 25. An object to be sterilized or disinfected is introduced into sterilization chamber 25 of housing 17 via entrance port 19 and leaves sterilization chamber 25 of housing 17 via exit port 21. Lamps 5, which emit light for sterilization or disinfection, are disposed inside housing 17, within sterilization chamber 25. Although lamps 5 are shown mounted on side walls 14 of housing 17, the lamps may be mounted on a front wall 16 and/or rear wall 18 of housing 17, or in any other location that allows for illumination of the object and/or sterilization chamber 25. Reflectors 7 are shown disposed about lamps 5 to direct the light emitted from lamps 5 towards sterilization chamber 25 and hence towards the object to be sterilized or disinfected, although such reflectors are not necessary. Reflectors may be provided in addition to or alternatively to reflectors 7 to direct and/or redistribute the light. For example, reflectors may be provided to direct light towards rollers 23 at exit port 21 to sterilize the surfaces of the exit rollers. Further, any of the interior surfaces of housing 17 or rollers 23 may be made from a reflective material (e.g., metal, reflective plastic) to redistribute the light emitted from lamps 5 and reduce energy loss due to light absorption.

It should be appreciated that while two lamps 5 are illustrated in FIG. 2A, any number of lamps may be used in accordance with the invention. Further, while lamps 5 are illustrated as generally linear in shape, lamps 5 may be round, ring-shaped, and/or comprised of a group of UV light-emitting devices. For example, a ring-shaped lamp may encircle the sterilization chamber 25. As discussed in connection with the embodiment of FIGS. 1A-1B, each of lamps 5 may be any light source that emits light capable of sterilization or disinfection (e.g., a mercury vapor lamp, a xenon flash lamp, a continuous arc lamp, UV light emitting diodes, a UV laser, or any other solid state or non-solid state UV light-emitting device), and may emit any spectrum of light suitable for sterilization or disinfection. The light may be generated either in flashes or as continuous radiation.

Rollers 23 are coupled to a motor 11 via a drive belt 13. Motor 11 may be controlled to cause drive belt 13 to rotate rollers 23 inward, as shown by arrows B. Rollers 23 at the entrance port 19 and exit port 21 may be driven at the same speed. Alternatively, rollers 23 at exit port 21 may rotate at a slightly higher speed than that of rollers 23 at entrance port 19 to maintain tension and ensure proper positioning of long devices (e.g., wires) as they move through housing 17. As discussed in connection with the embodiment of FIGS. 1A-1B, although motor 11 and drive belt 13 are described as providing movement to rollers 23, it should be appreciated that the invention is not limited to the use of a motor and drive belt for movement of the rollers. For example, other sources of rotational energy, e.g., a crank, may be used in place of motor 11, and other mechanisms for coupling the energy to drum 3, e.g., chains, gears, or rollers, may be used in place of drive belt 13.

Rollers 23 may have any of a variety of configurations, and may be shaped to accommodate a specific object. According to the illustrative embodiment of FIGS. 2A-2B, a pair of rollers 23 is provided at each of entrance port 19 and exit port 21. However, three or more rollers 23 may alternatively mate at each port, or a single roller may be provided at each port. In another example, rollers 23 may be entirely absent from exit port 21. Rollers 23 at each port, when present, may be pressed together to form a light seal between them. Housing 17 may align with the sides and ends of rollers 23 to form the remainder of the light seal. Baffles may be included on the sides of rollers 23 exposed to the light source within housing 17. The baffles may reduce the amount of damaging light absorbed by the rollers, and may thereby prolong the life of the rollers. The baffles may be light reflective to increase the efficiency of sterilization chamber 25.

Rollers 23 may be made from rubber, plastic, elastomeric or skinned foam, or any other suitable material. Preferably, rollers 23 are non-light transmissive to prevent light from escaping from housing 17, and compliant to accommodate the object for sterilization or disinfection. Rollers 23 may be smooth or may include extensions 27 to enhance compliance. Extensions 27, shown in FIG. 2A may be formed by making radial slits in the rollers. The compliance of the rollers can be further enhanced with axial slits to allow sections of the material to compress independently. A high degree of compliance in rollers 23 enables a complete light seal around objects having an inconsistent cross-sectional thickness.

Housing 17 may form a light-tight enclosure to prevent light exposure to an operator. Rollers 23 may seal entrance port 19 and exit port 21 and substantially prevent light from escaping via these ports. To enhance the seal, extensions 27 of a pair rollers 23 may be interleaved. A removable collection bag may be attached at the exit port to collect sterilized objected. The collection bag may be sterile to prevent contamination of the sterilized objects. Rollers 23 at exit port 21 may be omitted, and the collection bag may be used to form part of the light-seal of housing 17. The collection bag may be impermeable to light, and affixed to housing 17 to create a light-tight seal.

An object to be sterilized or disinfected is introduced into housing 17 via entrance port 19. In the example of FIGS. 2A-2B, the object enters between the two rollers 23 disposed at entrance port 19, which form a moving light seal. A sensor may be provided at entrance port 21 to detect the presence of an object. For example, an optical sensor may detect an object at entrance port 19 and cause rollers 23 to initiate rotation of rollers 23 and/or turn on lamps 5. The sensor may reduce the power consumption of the sterilizer/disinfector and extend the life of its components (e.g., lamps 5, motor 11, drive belt 13, bearings, etc.). Other sensors (e.g., mechanical or capacitive sensors) maybe used in place of an optical sensor.

The object then moves through sterilization chamber 25 in a continuous or intermittent motion and is disinfected or sterilized by light from lamps 5. Small objects (e.g., clips), will fall through sterilization chamber 25 after passing through rollers 23 at entrance port 19. Longer objects (e.g., wires or leads), portions of the object will simultaneously pass through rollers 23 at entrance port 19 and exit port 21. Light from lamps 5 in sterilization chamber 25 sterilize or disinfect the object as it passes through the chamber. Lamps 5 may flash light or continuously emit light at an intensity sufficient to achieve sterilization or disinfection of the object (e.g., 10 millijoules per square centimeter). One or more sensors may be disposed in sterilization chamber 25 and coupled to lamps 5. For example, an optical beam (not shown) may be disposed across sterilization chamber 25 to detect the presence of an object, and may trigger lamps 5 to turn on or flash when the optical beam is crossed.

A single flash with a few hundred microsecond duration from a xenon flash lamp may generate enough UV light to sterilize a falling object. During the flash, depending on its length, the falling object may move only a fraction of an inch. Multiple flashes may be produced during the duration of the fall, if desired. The output energy of lamps 5 and the speed of movement of the object through sterilization chamber 25 may be chosen to provide at least the minimum dosage for sterilization or disinfection on all points on the surface of the object to be sterilized, which is typically 10 millijoules per square centimeter of UV light energy. With a small sterilization chamber and a high-powered UV source, e.g., a xenon flash lamp operating at a high repetition rate, the speed of the object to be sterilized/disinfected may be several inches per second or faster. Larger or lower powered units may provide movement at a slower rate. The power of the UV source is chosen based on the surface area of the object and the dosage of UV light necessary for sterilization/disinfection.

The object may exit sterilization chamber 25 through rollers 23 at exit port 21, if present, which form another movable light seal. After exiting housing 17, the object may be released or may enter a collection container. Objects may be accumulated in the collection container. Alternatively, rollers 23 at exit port 21 may be removed and objects may be accumulated inside housing 17, where they may be later removed via an access door. Rollers 23 at exit port 21 may also be coupled to one or more sensors. For example, a sensor may detect when an object has reached rollers 23 at exit port 21 and cause lamps 5 to turn off. It should be appreciated that while exit port 21 is illustrated as separate from entrance port 19, a single port may alternatively be used, such that objects enter and exit through the same port in housing 17.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and equivalents thereto.

What is claimed is:

1. An apparatus for sterilizing or disinfecting an object having exterior surfaces, comprising:
a substantially light-tight housing;
at least one ultraviolet light-emitting lamp disposed within the housing; and
a drum to change the orientation of the object in the housing so that light emitted by the at least one lamp contacts substantially all of the exterior surfaces of the object.

2. The apparatus of claim 1, wherein a surface of the drum is continuous.

3. The apparatus of claim 2, wherein the surface of the drum is substantially impenetrable to light.

4. The apparatus of claim 1, wherein the at least one lamp is mounted to a wall of the housing that is interior to the drum.

5. The apparatus of claim 1, wherein the at least one lamp is disposed within the housing such that the at least one lamp emits light that contacts an interior wall of the drum and not an exterior wall of the drum.

6. The apparatus of claim 1, further including a timer, coupled to the drum, to control power to the drum.

7. The apparatus of claim 1, further including a door to provide access to an interior of the housing.

8. The apparatus of claim 1, further including a safety interlock, coupled to the door and the at least one lamp, to turn off the at least one lamp when the door is opened.

9. The apparatus of claim 1, wherein the at least one lamp is a flash lamp.

10. An apparatus for sterilizing or disinfecting an object having exterior surfaces, comprising:
a substantially light-tight housing;
at least one ultraviolet light-emitting lamp disposed within the housing; and
means for changing the orientation of the object in the housing so that light emitted by the at least one lamp contacts substantially all of the exterior surfaces of the object.

11. A batch process for sterilizing or disinfecting an object having exterior surfaces, comprising acts of:
enclosing the object within a substantially light-tight housing; and
changing the orientation of the object in the housing while irradiating the object with ultraviolet light, so that the ultraviolet light contacts substantially all of the exterior surfaces of the object.

12. The batch process of claim 11, further including acts of:
introducing the object into the housing via an opening; and
removing the object from the housing via the opening.

13. The batch process of claim 12, further including an act of closing a door on the housing, after introducing the object into the housing, to form a substantially light-tight seal at the opening.

14. The batch process of claim 11, wherein the act of changing the orientation of the object does not include any substantial lateral motion.

15. The batch process of claim 11, wherein the act of enclosing an object within a substantially light-tight housing includes placing an object in a substantially light-impermeable drum within the substantially light-tight housing, and further including an act of rotating the drum.

16. The batch process of claim 15, wherein the act of changing the orientation of the object includes changing the orientation of the object in the housing while irradiating the object with ultraviolet light from within the drum.

17. The batch process of claim 16, wherein the act of changing the orientation of the object further includes lifting the object and allowing it to fall while irradiating the object with ultraviolet light from within the drum.

18. The batch process of claim 11, wherein the act of enclosing an object within a substantially light-tight housing includes enclosing a medical device within the housing.

19. A method for sterilizing or disinfecting an object, comprising acts of:
introducing the object into a housing;
causing the object to move through the housing;
detecting the object with a sensor; and
activating a lamp based on a detection of the object with the sensor, the lamp illuminating the object as it moves through the housing.

20. The method of claim 19, wherein the act of causing the object to move through the housing includes causing the object to fall through the housing.

21. The method of claim 19, wherein the act of activating a lamp includes activating an ultraviolet light-emitting lamp.

22. The method of claim 19, wherein the act of introducing the object into a housing includes introducing a medical device into the housing.

23. An apparatus for sterilizing or disinfecting an object, comprising:
a housing having a light seal to substantially prevent light from exiting the housing;
at least one ultraviolet light-emitting lamp disposed within the housing; and
a mechanism for moving the object within the housing;
wherein the mechanism forms at least part of the light seal.

24. The apparatus of claim 23, wherein the mechanism is activated by a sensor.

25. The apparatus of claim 24, wherein the sensor is activated by presence or movement of the object.

26. The apparatus of claim 23, wherein the mechanism is one or more rollers.

27. The apparatus of claim 26, wherein the one or more rollers are interleaved.

28. The apparatus of claim 26, wherein the one or more rollers are light-reflective.

29. The apparatus of claim 23, wherein the mechanism for moving the object within the housing moves the object into the housing, and wherein the apparatus further includes a second mechanism for moving the object out of the housing.

30. The apparatus of claim 29, wherein the second mechanism is one or more rollers.

31. The apparatus of claim 23, wherein the housing has a reflective interior.

32. The apparatus of claim 23, wherein the at least one lamp is activated by a sensor.

33. The apparatus of claim 32, wherein the at least one lamp is activated by the object.

34. The apparatus of claim 33, wherein the sensor is an optical beam.
